(19) United States
(12) Patent Application Publication
(10) Pub. No.: US 2004/0230301 A1
(43) Pub. Date: Nov. 18, 2004

(54) Bioactive device having surface with alloyed layer of calcium phosphate compounds and method of making

(76) Inventors: Thomas D. Driskell, Westerville, OH (US); Arnold H. Deutchman, Columbus, OH (US)

Correspondence Address: John A. Haug, P.O. Box 386, West Harwich, MA, 02671, US

(21) Appl. No.: 10/877,629

(22) Filed: Jun. 26, 2004

Related U.S. Application Data
(63) Continuation-in-part of application No. 10/045,430, filed on Jan. 11, 2002.
(60) Provisional application No. 60/261,127, filed on Jan. 16, 2001.

(51) Int. Cl. A61F 2/02

(52) U.S. Cl. 623/11.11

(57) ABSTRACT

A dental or orthopedic implantable prosthetic device (1) which has a bioactive surface of an alloyed layer of material having calcium phosphate compounds. The device is formed by placing a suitable substrate of biocompatible material in a vacuum chamber (10), the substrate is cleaned by ion beam sputtering (18a) and then ion beam sputtering (14a) evolves and deposits (16a) bioactive material onto the surface of the device. The bioactive layer is mixed into the surface forming an alloyed zone by augmenting ion beam (18a) and is grown out to a selected thickness while being continuously bombarded by the augmenting ion beam.

### CROSS-REFERENCE TO RELATED APPLICATIONS

This is a continuation-in-part of Ser. No. 10/045,430, filed Jan. 11, 2002 which, under 35 U.S.C. Section 119(e)(1) claims the benefit of Provisional Application 60/261,127, filed Jan. 16, 2001.

### FIELD OF THE INVENTION

This invention relates generally to prosthetic devices including orthopedic, dental and other implantable devices and more particularly, to devices, such as implants, having a surface formed with improved bioactive characteristics.

### BACKGROUND OF THE INVENTION

Plasma sprayed hydroxylapatite (hereinafter also referred to as HA) coatings have been successfully used since at least the early 1980s to enhance the load transmitting capabilities of orthopedic and dental prosthetic implants placed into bone. Biocompatible materials, such as HA, have a unique attribute-compared to most so-called biomaterials in that they are “bioactive” and react compatibly with bone which forms a tenacious bond with HA, a phenomenon commonly known as biointegration. HA also has been demonstrated to enhance the speed of bone healing around implants. From the beginning, however, although widely used clinically, plasma sprayed HA coatings have been subject to a number of physical and biological phenomena that often compromise the health and even survival of the implant. A brief discussion of exemplary problematic areas follows.

Delamination of HA Coatings

The tenacity of the bond between plasma sprayed HA and titanium implant substrates can vary considerably due to processing variables. Even if the bond is good, the coating is still subject to chipping during surgical placement if the surgeon is not careful. Plasma sprayed HA implant surfaces exposed by chipping, or other processes of HA degradation, invariably appear to be grayish-black and rough as if burned.

Sub-Crestal Infections With Concomitant Bone Loss

Dental implants with plasma sprayed HA coating that extend supra-crestally into the gingival tissue appear to be more subject to infection than uncoated implants and can cause severe crestal bone loss as well as delamination and dissolution of the HA coating.

Infection often ensues, usually resulting in rapid degradation and loss of the HA coating in the vicinity of the infected area. These implants sometimes can be saved by reopening the implant site, debriding the infected area and abrading the exposed portions of the implant to remove the remaining supra-crestal HA coating down to a clean, bright titanium surface. If this salvage procedure is not attempted, the implant will probably be lost with a substantial loss of the surrounding bone.

### SUMMARY OF THE INVENTION

It is an object of the invention to overcome the prior art limitations noted above. Another object of the invention is to provide an orthopedic and dental prosthetic implant having improved bioactive characteristics.

Briefly stated, a prosthetic device made in accordance with a preferred embodiment of the invention has a surface formed with improved bioactive characteristics. According to a feature of the invention, an implantable device has a substrate of titanium alloy or other suitable biocompatible material with a layer of inorganic material comprising calcium phosphate containing compounds applied to the surface of the device. A preferred inorganic material for application to the surface is hydroxylapitite (HA). According to another feature of the invention, the layer is bombarded into the substrate using inert ions to form an alloy or intimate mixture of the substrate and inorganic materials. The alloyed surface can be overlaid with an inorganic surface layer continuously bombarded while grown to the alloyed surface, such as an HA type surface layer bonded to an apatitic titanium alloyed surface.

### BRIEF DESCRIPTION OF THE DRAWINGS

The Accompanying drawings, which are incorporated in and constitute a part of the specification, illustrate preferred embodiments of the invention, and together with the description serve to explain the objects, advantages and the principles of the invention.

FIG. 1 is a schematic view of apparatus used in making a prosthetic device according to the invention,

FIG. 2 is a chart showing the sequence of steps in forming the prosthetic device, and

FIG. 3 is a schematic elevational view of a broken away prosthetic device made in accordance with the invention.

Additional objects and features of the invention will be set forth in part in the description which follows and in part will be obvious from the description. The objects and advantages of the invention may be realized and attained by means of the instrumentalities pointed out in the appended claims.

### DETAILED DESCRIPTION OF THE PREFERRED EMBODIMENTS

In accordance with the invention, an orthopedic or dental prosthetic implant device is provided with a surface layer having a bioactive apatitic attribute permitting bone to bond directly and tenaciously to the implant. A preferred substrate material is commercially pure (C.P.) titanium or a titanium alloy such as Ti6Al4V and an implant made in accordance with the invention has a bioactive apatitic type titanium alloy (BATA) surface. However, other biocompatible implant material substrates can be used, such as other metals, ceramic and plastic. Alloy is used in the sense defined in Merriam-Webster's Collegiate Dictionary of a compound, mixture, or union of different things. Application parameters can vary the ratio of HA or other substances imbedded into the surface microstructure of the implant substrate. Further, the alloyed substrate surface, for example apatitic type titanium, can be overlaid with an HA type surface layer alloyed thereto. It is also within the purview of the invention to alloy other substances, alone or in combination, to alter or otherwise enhance the bioactive attributes and load transmitting abilities of the implant.

A preferred method of making an implant device in accordance with the invention employs a vacuum deposition process. The surfaces of an implant device are layered with a thin film of HA type material by means of a sputtering or any other thin film deposition technique. Simultaneously or sequentially, the HA type layered implant surface is bombarded with inert ions, such as argon, by means of a powerful ion beam accelerator device. The HA type material is impacted into the implant surface with such force by the inert material ions that it is driven into the interstitial spaces present in the microstructure of the implant substrate material, such as titanium. The process parameters are completely controllable. The depth of the alloyed apatitic type material titanium layer can be predetermined and the process additionally allows for an overlay application of an HA type layer, preferably between approximately 500 and 10,000 angstroms in thickness, intimately bonded to the underlying alloyed apatitic type material titanium alloy surface if desired. Thus, the HA type material literally becomes integrated with the implant material. One such process for obtaining the desired surface preparation can be adapted from the processes disclosed in U.S. Pat. No. 5,055,318, the subject matter of which is incorporated herein by this reference. Dimensionally, depth wise, these bioactive surfaces are measured in angstroms, with a suitable layer extending up to 5000 angstroms into the substrate material. Many types of materials can be alloyed with a variety of substrates using this process or variations of it. For example, a fluoroapatitic type titanium surface can be applied if desired. Another modification is to apply the BATA process onto a titanium plasma sprayed (TPS) particulate coated implant surface.

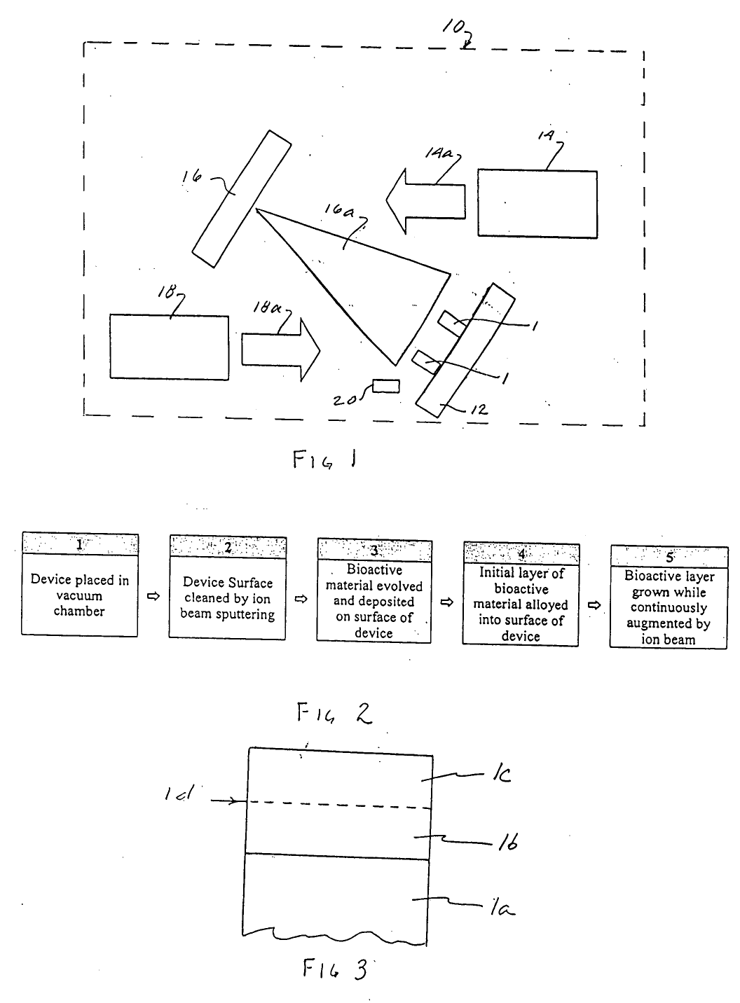

According to the preferred method of forming the improved prosthetic device surface, a dual ion beam process is employed and carried out in a vacuum chamber 10 indicated in a dashed line in FIG. 1. Substrates 1 to be treated are attached to a parts platen 12. A sputter ion source 14 directs a sputter beam 14a of inert gas ions toward target platen of bioactive material. Sputtered bioactive material 16a is directed toward devices 1 along with an augmenting inert gas ion beam 18a from augmenting ion source 18. A film thickness sensor 20 allows precise measurement of the thickness of the bioactive layer deposited in and on the surface of the devices.

TABLE I

| Feature | Function |
|---|---|
| Vacuum | Process is carried out in a high vacuum (allows control |
| Chamber | over the quality of the bioactive alloy formed in and |
| | on the surface of the device). |
| Sputter Ion | Inert gas ion beam sputters bioactive material from the |
| Beam | target platen |
| Target Platen | Bioactive material located on the target platen |
| Part Platen | Devices to be treated attached to the part platen |
| Augmenting | Inert gas ion beam used to first sputter clean the surface |
| Ion Beam | of the devices, next to mix the bioactive material into |
| | the surface of the devices forming the ballistically alloyed |
| | zone, then to control structure of the bioactive layer as |
| | it is grown out from the ballistically alloyed zone. |
| Film | Allows precise measurement of the thickness of the |
| Thickness | bioactive alloy layer deposited in and on the surface of |
| Sensor | the device. |

As noted above, the bioactive surfaces comprise alloyed layers of calcium phosphate compounds. Table I describes the general dual beam deposition process utilizing the FIG. 1 apparatus and FIG. 2 shows the processing sequence. As shown in FIG. 2, the devices are placed in a vacuum chamber at step 1; the surfaces of the devices are cleaned by ion beam sputtering at step 2; bioactive material is evolved and deposited on the surface of the devices at step 3; the initial layer of bioactive material is alloyed into the surface of the devices at step 4; and the bioactive layer is grown and continuously augmented by an ion beam at step 5. In FIG. 3 the device subsurface is shown schematically at 1a and the original surface of the device is indicated in a dashed line at 1d. The bioactive outer layer grown from a ballistically alloyed zone 1b is shown at 1c.

Table II includes specific individual steps in the processing sequence and identifies typical process parameters and ranges of parameters suitable for the process.

TABLE II

| Step | Description | Typical Process Parameters | Range Process Parameters |
|---|---|---|---|
| 1 | Device placed in vacuum chamber on | Vacuum: 1.0E(−07) Torr | Vacuum: 1.0E(−08) to |
| | an articulated fixture which allows | | 1.0E(−05) Torr |
| | programmed orientation of the device | | |
| | during the process. | | |
| 2 | Surface of device cleaned by ion beam | Ion Species: Ar | Ion Species: He, Ne, Ar, Kr |
| | sputtering with the ion beam from the | Beam Energy: 500 eV | or Xe |
| | augmenting ion source. | Beam Current: 1.0 mA/cm2 | Beam Energy: (0.1-100) keV |
| | | Time: 50 minutes | Beam Current: (0.01-1500) mA/cm2 |
| | | | Time: (0.033-5000) minutes |
| 3 | Sputter ion beam use to ion beam | Ion Species: Ar | Ion Species: He, Ne, Ar, Kr or |
| | sputter hydroxylapatite or other | Beam Energy: 1000 eV | Xe |
| | bioactive material from the target plate | Beam Current: 2.5 mA/cm2 | Beam Energy: (0.1-100) keV |
| | onto the surface of device. | Material: hydroxylapatite | Beam current: (0.1-1500) mA/cm2 |
| | | Evolution Rate: 0.2 angstom/sec | Material: apatitic minerals |
| | | | including calcium and/or |
| | | | phosphorous containing |
| | | | compounds, or fluoride |
| | | | containing compounds |
| | | | including Ca2F |
| | | | Evolution Rate: (0.008-120) angstoms/sec. |
| 4 | Augmenting ion beam used to | Ion Species: Ar | Ion Species: He, Ne, Ar, Kr or |
| | ballistically alloy first few layers of | Beam Energy: 1000 eV | Xe |
| | suttered bioactive material into device | Beam Current: 1.0 mA/cm2 | Beam Energy: (0.1-100) keV |
| | surface. | Thickness: 2,000 angstoms | Beam Current: (0.1-1500) mA/cm2 |
| 5 | Bioactive layer is grown out from the | Ion Species: Ar | Ion Species: He, Ne, Ar, Kr or |
| | ballistically alloyed layer as ion beam | Beam Energy: 200 eV | Xe |
| | sputtering of the target continues. | Beam Current: 0.05 mA/cm2 | Beam Energy: (0.1-100) keV |
| | Augmenting ion beam used to control | Thickness: 2,000 angstoms | Beam Current (0.01-1500) mA/cm2 |
| | the structure of the bioactive layer as is | | Thickness: (100-100,000) |
| | grown. | | angstroms |

Table III includes the Table II steps and step descriptions along with actual parameters for examples of carrying out the process identified as Run #1 and Run #2, resulting in a coating thickness of 2,265 angstroms for the devices of Run #1 and 2,812 angstroms for the devices of Run #2.

TABLE III

| Step | Step Description | Run #1 | Run #2 |
|---|---|---|---|
| 1 | Device placed in vacuum | Vacuum: 5.0E(−05) Torr | Vacuum: 7.0E(−05) Torr |
| | chamber on an articulated fixture | | |
| | which allows programmed | | |
| | orientation of the device during | | |
| | the process. | | |
| 2 | Surface of device cleaned by ion | Ion Species: Ar | Ion Species: Ar |
| | beam sputtering with the ion | Beam Energy: 500 eV | Beam Energy: 500 eV |
| | beam from the augmenting ion | Beam Current: 7.0 mA/cm2 | Beam Current: 6.0 mA/cm2 |
| | source. | Time: 50 minutes | Time: 50 minutes |
| 3 | Sputter ion beam use to ion | Ion Species: Ar | Ion Species: Ar |
| | beam sputter hydroxylapatite or | Beam Energy: 1000 eV | Beam Energy: 1000 eV |
| | other bioactive material from the | Beam Current: 1.0 mA/cm2 | Beam Current: 1.0 mA/cm2 |
| | target platen onto surface of | Material: hydroxylapatite | Material: hydroxylapatite |
| | device | Evolution Rate: 0.2 angstom/sec | Evolution Rate: 0.2 angstom/sec |
| 4 | Augmenting ion beam used to | Ion Species: Ar | Ion Species: Ar |
| | ballistically alloy first few layers | Beam Energy: 1000 eV | Beam Energy: 1000 eV |
| | of sputtered bioactive material | Beam Current: 1.0 mA/cm2 | Beam Current: 1.0 mA/cm2 |
| | onto device surface. | Time: 12 minutes | Time: 15 minutes |
| 5 | Bioactive layer is grown out | Ion Species: Ar | Ion Species: Ar |
| | from the ballistically alloyed | Beam Energy: 200 eV | Beam Energy: 200 eV |
| | layer as ion beam sputtering of | Beam Current: 0.05 mA/cm2 | Beam Current: 0.05 mA/cm2 |
| | the target continues. | Thickness: 2,265 angstoms | Thickness: 2,812 angstoms |
| | Augmenting ion beam used to | | |
| | control the structure of the | | |
| | bioactive layer as it is grown | | |

Virtually all of the previously mentioned problems associated with plasma sprayed HA implants are resolved by use of the BATA surface technique described above for the following reasons. There is no oxidation or other high temperature deterioration (blackening) of the implant substrate from the BATA process. The previously noted problems are eliminated because no appreciable heat is applied during the BATA surface application process. Delamination or chipping is eliminated due to the alloyed nature of the BATA surface which is integrated into the implant substrate. However, abrasion or other marring of the BATA surface is still possible through careless handling during surgical placement. The clinical significance however, is of no greater importance than the same type of abrasion on a conventional uncoated or TPS coated titanium implant.

Preliminary in-vivo studies in dog femurs indicates histologically that advanced healing of surrounding bone abutting the BATA surface test specimens was evident at six weeks when compared to uncoated control specimen seen at twelve weeks.

X-ray diffraction has been classically used for crystallographic assessment of bulk materials. This technique has also been modified in order to enable evaluation of the top outermost layers of materials and has been widely utilized for surface characterization. Devices made in accordance with the invention were subjected to this modified technique and the result of the procedure revealed an amorphous calcium and phosphate based layer along with alpha titanium.

This surface modification, that is, the alloyed surface of intermixed amorphous calcium and phosphate with titanium, results in a higher dissolution rate and enhanced bioactivity than that of crystalline phases.

Although the invention has been described with regard to specific preferred embodiments thereof, variations and modifications will become apparent to those skilled in the art. For example, additional inorganic material, such as calcium fluoride, can be alloyed into the HA overlayer coating in accordance with the teaching of the invention, it is, therefore, the intent that the appended claims be interpreted as broadly as possible in view of the prior art to include all such variations and modifications.

What is claimed:

1. A device for implantation in a living body, the device having enhanced bioactivity to promote early osseointegration with bone structure into which the device is implanted comprising:
a substrate of biocompatible material, the substrate having an outer surface and a thin layer of calcium and phosphate in the amorphous phase alloyed with the biocompatible material of the substrate to a selected depth.

2. A device according to claim 1 in which the substrate material is selected from the group consisting of titanium alloy and commercially pure titanium.

3. A device according to claim 2 in which the calcium and phosphate is alloyed with the substrate material to a depth of approximately 5,000 angstroms.

4. A device according to claim 1 further comprising an outer layer of a calcium phosphate containing compound.

5. A device according to claim 4 in which the outer layer has a thickness within the range of approximately 2,000 to 3,000 angstroms.

6. A method of enhancing the bioactivity of a device for implantation in a living body to promote early osseointegration comprising the steps of
taking a substrate of biocompatible material,
applying a thin layer of a calcium phosphate containing compound to the substrate,
alloying the thin layer with the biocompatible material up to a depth of approximately 5,000 angstroms in which the thin layer is in the amorphous phase thereby promoting early osseointegration.

7. The method of claim 6 in which the substrate material is selected from the group consisting of titanium alloy and commercially pure titanium.

8. The method of claim 7 in which the thin layer is alloyed with the biocompatible material by bombarding the substrate with inert ions.
